(19) United States
(12) Patent Application Publication
(10) Pub. No.: US 2020/0253579 A1
(43) Pub. Date: Aug. 13, 2020

(54) ULTRASONIC MEASUREMENT OF VESSEL STENOSIS

(71) Applicant: KONINKLIJKE PHILIPS N.V., EINDHOVEN (NL)

(72) Inventor: JAMES ROBERTSON JAGO, SEATTLE, WA (US)

(21) Appl. No.: 16/758,586

(22) PCT Filed: Oct. 16, 2018
(86) PCT No.: PCT/EP2018/078130

§ 371 Date: Apr. 23, 2020

Related U.S. Application Data
(60) Provisional application No. 62/576,235, filed on Oct. 24, 2017.

(51) Int. Cl. A61B 8/08; G01S 15/89; A61B 8/06

(52) U.S. Cl. CPC A61B 8/085; G01S 15/8993; A61B 8/483; A61B 8/5223; A61B 8/488; A61B 8/5246; A61B 8/06

(57) ABSTRACT

An ultrasound system is used to measure the percent stenosis of a vessel in terms of residual lumen area. A measurement of volume blood flow is made at an unobstructed point of the vessel near the site of the stenosis. A measurement of the time averaged mean blood flow velocity is made at the stenosis. The quotient of these two values is computed to produce an estimate of the residual lumen area and the percent stenosis at site of the obstruction.

This invention relates to medical diagnostic ultrasound systems and, in particular, to the use of ultrasound systems to measure vessel stenosis, the percentage occlusion of a blood vessel.

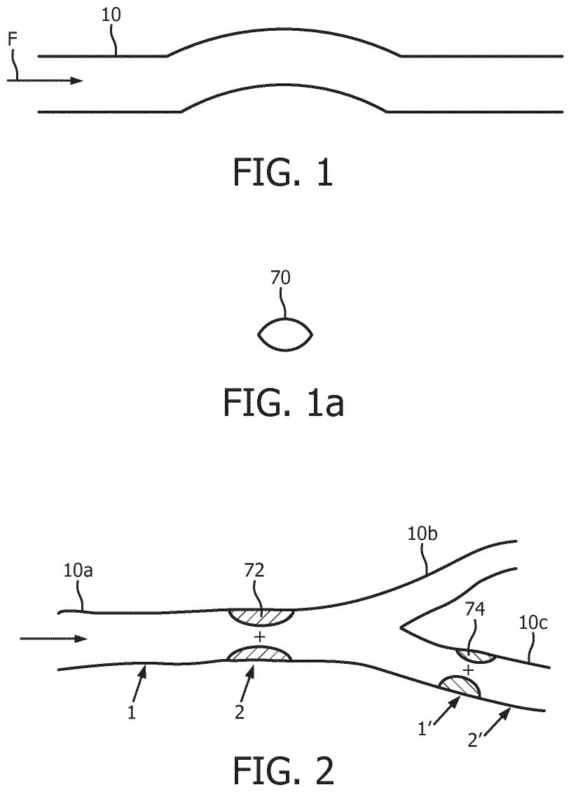

The obstruction of blood vessels by the buildup of plaque and other substances can prevent the flow of an adequate supply of nourishing blood to tissues and organs in the body. Hence it is desirable to be able to detect and measure blood vessel obstructions, generally as a percent stenosis, the percentage reduction of the normal flow lumen caused by the plaque. Visualizing and measuring the obstruction with ultrasound is problematic with two-dimensional (2D) ultrasound due to the difficulty in obtaining the correct image plane for the proper measurement. Three dimensional (D) ultrasound will obviate this problem, but is nonetheless hampered by shadowing from the plaque calcification and insufficient resolution. The most common way to quantify vessel obstruction is not by ultrasound, but by angiography. Since angiograms are projection images, they are useful for assessing vessel diameter reduction and not flow lumen area change. FIG. 1 illustrates a difficulty in assessing lumen size with projection images. In FIG. 1 blood is flowing in blood vessel 10 as indicated by flow vector F. Vessel 10 is completely unobstructed in this example, but has a bend as shown in the drawing. If a projection image were taken parallel to the flow direction F, the resultant image of the lumen would appear as shown by lumen 70 in FIG. 1a. Thus, this view of the vessel 10 could be taken to be that of an obstructed vessel. Angiograms are not normally taken parallel to the flow direction as in FIG. 1, but normal to the length of the vessel as FIG. 1 is viewed, but the same principle of reconstruction applies. The resultant angiogram will be strongly affected by the rotational orientation of the plaque within the vessel and the tortuous path of the vessel, and for these reasons numerous angiograms are normally acquired at different look directions to the vessel. By comparing different views, an assessment of the degree of stenosis is made, typically using the NASCET standard which relates the perceived residual lumen diameter at the stenosis to the diameter of the vessel lumen at an unobstructed point in the vessel. Even with multiple views of a vessel, however, degree of stenosis is often underestimated with angiography. Nonetheless, such measurements are preferred over the current ultrasonic method for assessing stenosis, which is to measure the peak blood flow velocity at a stenosis, then relate this velocity to a vessel diameter reduction based on known previous measurements. But ultrasound is simple and easy to use, and does not involve the use of radiographic contrast agents as does angiography. Thus it would be desirable to be able to use ultrasound to perform initial assessment of vessel stenosis if an accurate and reliable ultrasonic technique were available. It would further be desirable for such assessment to measure lumen area reduction rather than diameter reduction, as it has been found that the hemodynamic effects of stenosis are more closely related to residual lumen area rather than diameter.

In accordance with the principles of the present invention, an ultrasound system and ultrasonic measurement technique are described for measuring the percent stenosis of a vessel in terms of lumen area reduction. A measurement of volume blood flow is made at an unobstructed point of the vessel proximal the site of the obstruction. A measurement of the blood flow velocity is made at the stenosis. The quotient of these two values is computed to produce an estimate of the residual lumen area and the percent stenosis at site of the obstruction. The volume blood flow measurement is preferably made using 3D ultrasound.

In the drawings:

FIG. 1 illustrates a tortuous unobstructed blood vessel.

FIG. 1a illustrates a projection image of the lumen of the blood vessel of FIG. 1 taken in the direction of the blood flow.

FIG. 2 illustrates a carotid artery with stenotic regions in the common carotid artery and the internal carotid artery which are to be measured for percent stenosis in accordance with the principles of the present invention.

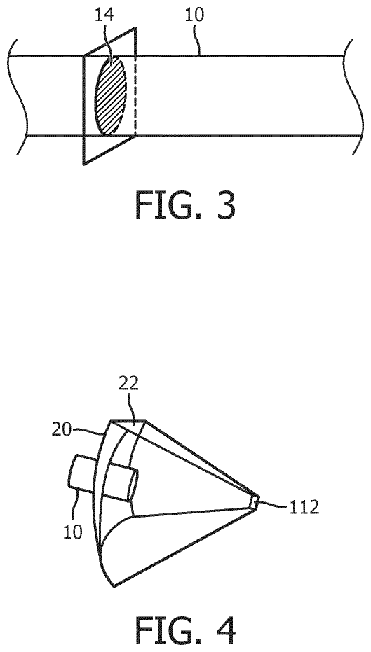

FIG. 3 illustrates a blood vessel with a cross sectional area where volume blood flow is to be measured.

FIG. 4 illustrates the measurement of volume blood flow through a virtual surface in front of an ultrasound transducer.

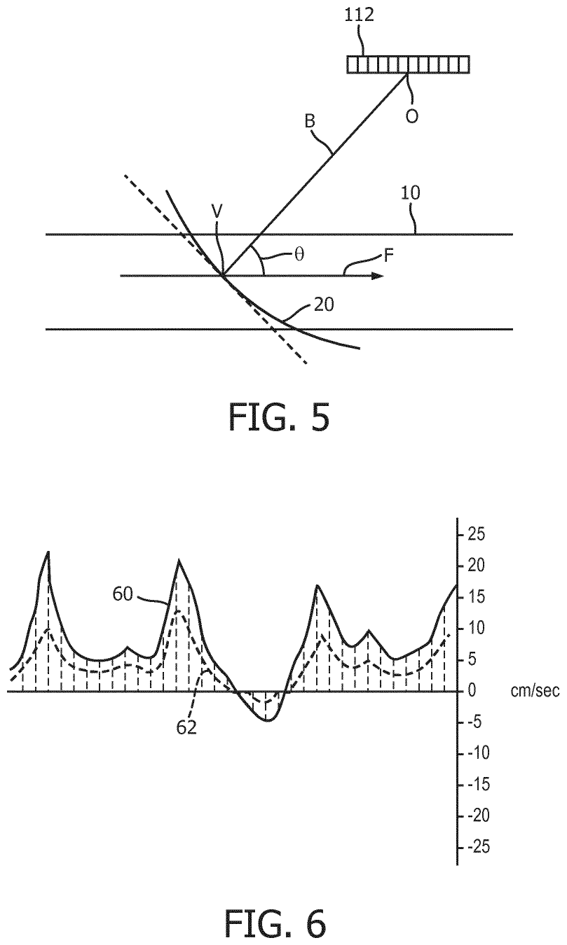

FIG. 5 illustrates why no angle correction is needed for the volume flow measurement technique of FIG. 4.

FIG. 6 illustrates a spectral Doppler display with a tracing of its mean velocity over several heart cycles.

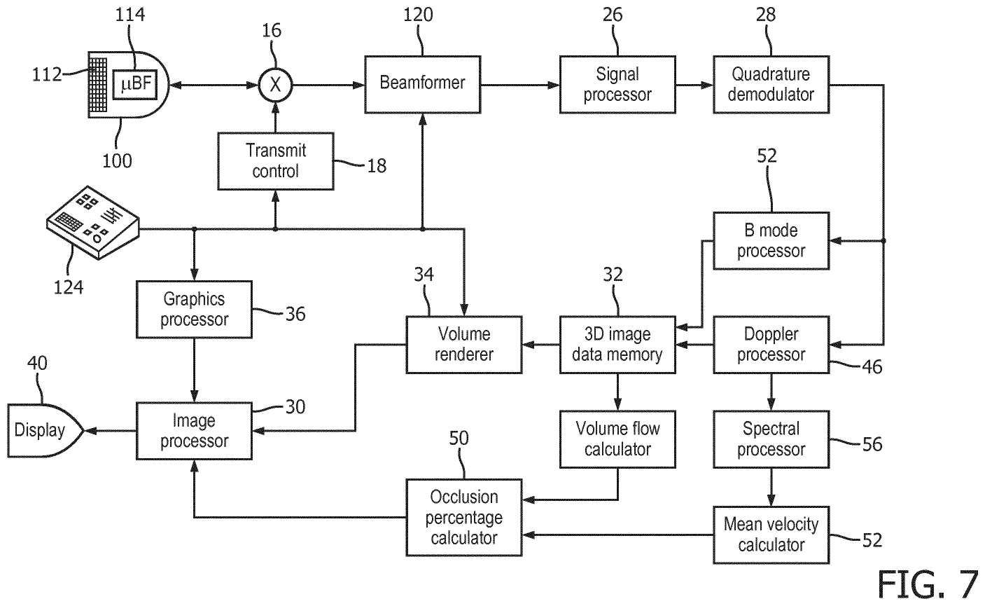

FIG. 7 is a block diagram of an ultrasound system constructed in accordance with the principles of the present invention.

Referring to FIG. 2, three sections of a branching carotid artery are illustrated, the common carotid artery 10a, the external carotid artery 10b, and the internal carotid artery 10c. Plaque buildup can occur in the carotid artery, restricting the flow of blood to the brain, and this example illustrates two such areas: an obstruction 72 in the common carotid artery and an obstruction 74 in the internal carotid artery. It is desired to measure the percent stenosis caused by these two obstructions. In accordance with the principles of the present invention, a volume flow measurement is taken at an unobstructed point in an obstructed artery and a flow velocity measurement is taken at the stenosis. These two values are then used to calculate percent area reduction of the artery caused by the stenosis. These measurements are premised on the fact that volume flow of blood Q through a cross-section of an artery is equal to the time average velocity V of blood flow times the area A of the cross-section, or

Q=v·A [1]

In the case of the common carotid artery obstruction, a volume flow measurement is taken at the unobstructed point indicated by the circled “1”. At this point in the artery,

Q₁=v₁·A₁ [2]

where A₁ is the unobstructed cross-sectional area at this point in the vessel. Since all of the blood flowing through the vessel at point 1 will then flow through the obstruction at the circled “2”, it is known that

Q₁=Q₂ [3]

A time average velocity measurement is now taken at the stenosis at point 2 in the vessel. This may be done using spectral Doppler and measuring the time averaged mean velocity of the blood flow through the stenosis. The user positions a Doppler sample volume cursor over the narrow obstruction of the stenosis as shown by the “+” icon in the drawing, then starts the Doppler acquisition to measure velocity at this point in the vessel. At the stenosis it is known that

Q₂=v₂·A₂ [4]

where Q₂ is the volume flow of blood through the stenotic point 2 and A₂ is the area of the residual lumen at the stenosis, the reduced area it is desired to measure. Since it is known that Q₁=Q₂ and the blood flow velocity v₂ at the stenosis has been measured by spectral Doppler, the area of the residual lumen is computed by

$${A}_{2} = \frac{{v}_{2}}{{Q}_{2}} = \frac{{v}_{2}}{{Q}_{1}} \qquad [ 5 ]$$

and the percent reduction of the area of the lumen of the vessel is

$$100 \times ( 1 - \frac{{A}_{2}}{{A}_{1}} ) \qquad [ 6 ]$$

In the internal carotid artery in FIG. 2, an obstruction is at circled point “1′”. The volume flow measurement previously made in the common carotid artery cannot be used to measure the percent stenosis in the internal carotid artery because the blood flow of the CCA splits, with some passing into the ECA and the rest flowing in the ICA. Thus, the volume flow measurement for this second obstruction must be taken in the ICA where all of the blood flowing through the obstruction at point 1′ also flow through the vessel at the measurement point, which is the circled “2′” in this example. A volume flow measurement is taken at point 2, and a time average velocity measurement is taken at the stenosis as indicated by the “+” icon. Then the area of the residual lumen at the stenosis is computed as explained above.

With reference to FIG. 3, the volume flow rate of blood through a blood vessel 10 can be measured by measuring the volume flow rate through any arbitrary sample surface 14 passing through the vessel. The volume flow rate through the sample surface 14 can be measured by first determining the velocity of blood flowing through the sample surface 14 by performing a three-dimensional Doppler scan. The velocity is then integrated throughout the area of the sample surface 14.

The sample surface 14 can be of any arbitrary shape or orientation. The reason the surface 14 need not be particularly oriented is that whatever volume of blood flows through the vessel 10 also flows through the sample surface 14. Thus, the sample surface 14 can be any arbitrary shape having any arbitrary orientation to the flow of blood through the vessel 10. In a preferred implementation of the present invention, a spherical sample surface 20 is obtained by obtaining a three-dimensional Doppler image in a narrow sample volume 22 equidistant from a two-dimensional array transducer 112 as shown in FIG. 4. A Doppler scan of this type is in this context referred to as Flow-mode, or F-mode, scanning. A 3-D flow image is obtained by an F-mode scan and is rendered with a spherical cross section 20 through the blood vessel 10, and the velocity values on the virtual spherical surface 20 are integrated to obtain the volume flow measurement as described more fully below.

The Doppler flow at points on the virtual spherical surface 20 is sampled by transmitting beams B steered from a common origin O of the two-dimensional array 112 as shown in FIG. 5. The echo signals at a common depth V along each beam are acquired to thereby acquire echoes on the spherical which intersects the blood vessel 10. The spherical surface is thus normal to the beam at each sampling point V. In instances where the two-dimensional is not square but is rectangular, the virtual surface can be toroidal in shape, but can be used to the same effect. The acquired signals at points V of the beams B will be echoes from blood flow for each point V which is inside the lumen of the blood vessel 20, and will be returned from tissue at points in the vessel wall and surrounding tissue. The flow signal can therefore be segmented by a Doppler wall filter as is known in the art. To account for boundary effects where echoes are returned from points near the vessel wall, and are thus likely to be a mix of flow and tissue signals, the returning echoes can be weighted by the intensity of the power Doppler characteristic of each echo, thereby weighting signals from the lumen boundary less than those more in the interior of the vessel. Normally, the measured Doppler velocity values on the surface are angle-dependent and need to be scaled as a function of the cosine of the incident Doppler angle, the angle between the Doppler beam B and the direction of flow F. But since the Doppler beam B is perpendicular to the unit area of the surface 20 at the sampling point V, as indicated by the dashed line demarcating the plane of the unit area, the angle between the perpendicular to the unit area and the flow direction has the same cosine term as the Doppler angle θ. Thus, Gauss's law for volume flow results in cancellation of the two cosine terms and no scaling of the measured velocity values is needed prior to summation (integration) of the velocity values in the lumen.

FIG. 6 illustrates a typical spectral Doppler display produced by an ultrasound system. The abscissa is calibrated in cm/sec and the ordinate is a time axis. Each vertical line is a measure of the spread of velocities at the sample volume in the subject from which the Doppler signals are acquired, e.g., the + icon in FIG. 3, at the time of acquisition. The peak velocity values are traced from one spectral line to the next by a trace 60, and the mean velocity values are connected by a dashed line 62. The acquisition and display of the Doppler spectrum of FIG. 6 is detailed in U.S. Pat. No. 5,606,972 (Routh). In an implementation of the present invention it is preferred to use a time averaged mean velocity value for the blood flow velocity value at a stenosis, which is obtained by averaging the mean velocity values on dashed line 62 over the interval of a heart cycle.

Referring to FIG. 7, an ultrasound system constructed for measuring the area reduction of a vessel due to a stenosis in accordance with the present invention is shown in block diagram form. A transducer array 112 is provided in an ultrasound probe 100 for transmitting ultrasonic waves and receiving echo information over a volumetric region of a body. The transducer array 112 may be a two-dimensional array of transducer elements capable of electronically scanning in two or three dimensions, in both elevation (in 3D) and azimuth, as shown in the drawing. Alternatively, the transducer may be a one-dimensional array of elements capable of scanning image planes which is oscillated back and forth to sweep the image plane through a volumetric region and thereby scan the region for three-dimensional imaging, such as that described in U.S. Pat. No. 7,497,830 (Li et al.) A two-dimensional transducer array 112 is coupled to a microbeamformer 114 in the probe which controls transmission and reception of signals by the array elements. Microbeamformers are capable of at least partial beamforming of the signals received by groups or “patches” of transducer elements as described in U.S. Pat. No. 5,997,479 (Savord et al.), U.S. Pat. No. 6,013,032 (Savord), and U.S. Pat. No. 6,623,432 (Powers et al.) The microbeamformer is coupled by the probe cable to a transmit/receive (T/R) switch 16 which switches between transmission and reception and protects the main system beamformer 120 from high energy transmit signals. The transmission of ultrasonic beams from the transducer array 112 under control of the microbeamformer 114 is directed by a transmit controller 18 coupled to the T/R switch and the beamformer 120, which receives input from the user's operation of the ultrasound system user interface or controls 124. Among the transmit characteristics controlled by the transmit controller are the spacing, amplitude, phase, and polarity of transmit beams and waveforms. Beams formed in the direction of pulse transmission may be steered straight ahead from the transducer array, or at different angles for a wider sector field of view or to scan a volumetric region such as that in front of transducer array 112 and including spherical surface 20 in FIG. 4.

The echoes received by a contiguous group of transducer elements are beamformed by appropriately delaying them and then combining them. The partially beamformed signals produced by the microbeamformer 114 from each patch of transducer elements are coupled to a main beamformer 120 where partially beamformed signals from individual patches of transducer elements are delayed and combined into a fully beamformed coherent echo signal. For example, the main beamformer 120 may have 128 channels, each of which receives a partially beamformed signal from a patch of 12 transducer elements. In this way the signals received by over 1500 transducer elements of a two-dimensional array transducer can contribute efficiently to a single beamformed signal.

The coherent echo signals undergo signal processing by a signal processor 26, which includes filtering by a digital filter and noise reduction as by spatial or frequency compounding. The digital filter of the signal processor 26 can be a filter of the type disclosed in U.S. Pat. No. 5,833,613 (Averkiou et al.), for example. The processed echo signals are demodulated into quadrature (I and Q) components by a quadrature demodulator 28, which provides signal phase information and can also shift the signal information to a baseband range of frequencies.

The beamformed and processed coherent echo signals are coupled to a B mode processor 52 which produces a B mode image of structure in the body such as tissue. The B mode processor performs amplitude (envelope) detection of quadrature demodulated I and Q signal components by calculating the echo signal amplitude in the form of (I²+Q²)^{1/2}. The quadrature echo signal components are also coupled to a Doppler processor 46, which stores ensembles of echo signals from discrete points in an image field which are then used to estimate the Doppler shift at points in the image, e.g., the points on a virtual spherical surface intersecting a blood vessel, with a fast Fourier transform (FFT) processor. The Doppler shift is proportional to motion at points in the image field, e.g., blood flow and tissue motion. For a color Doppler image, a surface of which may be used for the volume flow measurement, the estimated Doppler flow values at each point on the virtual surface 20 through a blood vessel are wall filtered and the surface Doppler values used to produce the volume flow measurement as described above. The surface Doppler values and others throughout a scanned volume may also be converted to color values using a look-up table to produce a colorflow Doppler image. Either the B mode image or the Doppler image may be displayed alone, or the two shown together in anatomical registration in which the color Doppler overlay shows the blood flow in tissue and in vessels in the imaged region.

The B mode image signals and the Doppler flow values are coupled to a 3D image data memory 32, which stores the image data in x, y, and z addressable memory locations corresponding to spatial locations in a scanned volumetric region of a subject. This volumetric image data is coupled to a volume renderer 34 which converts the echo signals of a 3D data set into a projected 3D image as viewed from a given reference point as described in U.S. Pat. No. 6,530,885 (Entrekin et al.) The reference point, the perspective from which the imaged volume is viewed, may be changed by manipulation of a control on the control panel 124, which enables the volume to be tilted or rotated to observe the scanned region from different viewpoints. The volume rendered image is coupled to an image processor 30 for display on a display 40.

In accordance with the principles of the present invention, the Doppler signal samples acquired from the sample volume+at the stenosis are coupled to a spectral Doppler display processor 56. The mean velocity values traced on each spectral line as shown in FIG. 6 are averaged over the interval of a heart cycle by a mean velocity calculator 52 to produce a time averaged mean velocity value of the blood flow at the stenosis which is coupled to an occlusion percentage calculator 50. The Doppler flow velocity values acquired at points on the virtual surface 20 which are stored in the 3D image data memory 32 are coupled to a volume flow calculator 54 which sums (integrates) the velocity values to produce a volume flow value, which is coupled to the occlusion percentage calculator. The occlusion percentage calculator computes the quotient of the time averaged mean velocity value at the stenosis and the volume flow measurement at the unoccluded point in the vessel to compute the residual flow lumen area at the stenosis using equation [5] above. The percent area reduction (occlusion percentage) may also be computed by the occlusion percentage calculator using equation [6]. The area A₁ of the unoccluded lumen, area 14 in FIG. 3, may be calculated by segmenting out the lumen area normal to the direction of flow from the colorflow volume image by multiplanar reconstruction, and measuring the area using known techniques, as ultrasound image data is calibrated to be anatomically accurate. Alternately, A₁ may be calculated by making one or more velocity measurements at the unoccluded point in the lumen to compute v₁ and using the volume flow measurement to compute A₁ using equation [2]. The residual lumen area at the stenosis and/or the percentage stenosis values are coupled to the image processor or a graphics processor 36 for display on image display 40. The graphics processor may also be employed if desired to illustrate the virtual surface 20 in registration with the 3D ultrasound image on the display.

It should be noted that an ultrasound system suitable for use in an implementation of the present invention, and in particular the component structure of the ultrasound system of FIG. 7, may be implemented in hardware, software or a combination thereof. The various embodiments and/or components of an ultrasound system, for example, the processors, calculators, and volume renderer of FIG. 7, or components, processors, and controllers therein, also may be implemented as part of one or more computers or microprocessors. The computer or processor may include a computing device, an input device, a display unit and an interface, for example, for accessing the Internet. The computer or processor may include a microprocessor. The microprocessor may be connected to a communication bus, for example, to access a PACS system or the data network for importing training images. The computer or processor may also include a memory. The memory devices such as the 3D image data memory and those used to store Doppler ensembles may include Random Access Memory (RAM) and Read Only Memory (ROM). The computer or processor further may include a storage device, which may be a hard disk drive or a removable storage drive such as a floppy disk drive, optical disk drive, solid-state thumb drive, and the like. The storage device may also be other similar means for loading computer programs or other instructions into the computer or processor.

As used herein, the term “computer” or “module” or “processor” or “workstation” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of these terms.

The computer or processor executes a set of instructions that are stored in one or more storage elements, in order to process input data. The storage elements may also store data or other information as desired or needed. The storage element may be in the form of an information source or a physical memory element within a processing machine.

The set of instructions of an ultrasound system including those controlling the acquisition, processing, and transmission of ultrasound images as described above may include various commands that instruct a computer or processor as a processing machine to perform specific operations such as the methods and processes of the various embodiments of the invention. The set of instructions may be in the form of a software program. The software may be in various forms such as system software or application software and which may be embodied as a tangible and non-transitory computer readable medium. Further, the software may be in the form of a collection of separate programs or modules such as a neural network model module, a program module within a larger program or a portion of a program module. The software also may include modular programming in the form of object-oriented programming. The processing of input data by the processing machine may be in response to operator commands, or in response to results of previous processing, or in response to a request made by another processing machine.

Furthermore, the limitations of the following claims are not written in means-plus-function format and are not intended to be interpreted based on 35 U.S.C. 112, sixth paragraph, unless and until such claim limitations expressly use the phrase “means for” followed by a statement of function devoid of further structure.

1. An ultrasonic diagnostic imaging system for assessing the degree of stenosis of a vessel caused by an obstruction, the ultrasonic diagnostic imaging system comprising:
an ultrasound probe adapted to acquire three-dimensional ultrasound data from blood flow in the vessel;
a 3D data memory coupled to the ultrasound probe, and adapted to store the three-dimensional ultrasound data from blood flow in the vessel;
a volume flow calculator, coupled to the 3D data memory, and adapted to compute a volume flow measurement at an unobstructed point of the vessel;
a Doppler processor, coupled to the ultrasound probe and responsive to ultrasound data from blood flow in the vessel, and adapted to produce a velocity measurement at the stenosis of the vessel; and
an occlusion calculator, responsive to the volume flow measurement and the velocity measurement, and adapted to produce a measurement of the flow reduction caused by the stenosis based on a quotient of the velocity measurement at the stenosis of the vessel and the volume flow measurement at the unobstructed point of the vessel.

2. The ultrasonic diagnostic imaging system of claim 1, wherein the occlusion calculator is further adapted to produce a measurement of the degree of stenosis of the vessel.

3. The ultrasonic diagnostic imaging system of claim 1, wherein the occlusion calculator is further adapted to produce a measurement of the percent reduction of the area of a lumen of the vessel caused by the stenosis.

4. The ultrasonic diagnostic imaging system of claim 1, wherein the occlusion calculator is further adapted to produce a measurement of the area of a residual lumen of the vessel.

5. The ultrasonic diagnostic imaging system of claim 1, wherein the volume flow calculator is further adapted to calculate the sum or integral of velocity values of a virtual surface intersecting the vessel and
wherein the virtual surface is obtained by obtaining a three-dimensional Doppler image in a narrow sample volume equidistant from a two-dimensional array transducer accommodated in the probe.

6. The ultrasonic diagnostic imaging system of claim 5, wherein the virtual surface further comprises a spherical virtual surface.

7. The ultrasonic diagnostic imaging system of claim 5, wherein the virtual surface further comprises a toroidal virtual surface.

8. The ultrasonic diagnostic imaging system of claim 5, wherein the volume flow calculator is further adapted to calculate the sum or integral of Doppler velocity values located on a virtual surface intersecting the vessel.

9. The ultrasonic diagnostic imaging system of claim 5, wherein the velocity values are weighted in proportion to power Doppler values calculated for locations corresponding to locations of the velocity values in the vessel.

10. The ultrasonic diagnostic imaging system of claim 1, wherein the Doppler processor further comprises a spectral Doppler processor.

11. The ultrasonic diagnostic imaging system of claim 10, wherein the spectral Doppler processor further comprises a time averaged mean velocity calculator.

11. The ultrasonic diagnostic imaging system of claim 11, wherein the occlusion calculator is further adapted to compute the quotient of a time averaged mean velocity value and a volume flow measurement.

13. A method for ultrasonically measuring the degree of stenosis at a point in a vessel comprising:
measuring volume flow at an unoccluded point in the vessel;
measuring flow velocity at an occluded point in the vessel;
computing area of stenosis at the occluded point using the volume flow measurement and the flow velocity measurement.

14. The method of claim 13, wherein measuring volume flow further comprises summing or integrating velocity values of a virtual surface intersecting the vessel.

15. The method of claim 13, wherein measuring flow velocity further comprises measuring time averaged mean velocity at the occluded point by spectral Doppler analysis.
